(19) United States
(12) Patent Application Publication
(10) Pub. No.: US 2021/0322725 A1
(43) Pub. Date: Oct. 21, 2021

(54) A MEDICAL DEVICE FOR DEPLOYING A CATHETHER THAT CAN BE OPERATED WITH A SINGLE HAND, AND A METHOD

(71) Applicant: Wake Forest University Health Sciences, Winston Salem, NC (US)

(72) Inventors: Doug Jaffe, Lewisville, NC (US); Austin Sellers, Dunedin, FL (US)

(21) Appl. No.: 17/048,879

(22) PCT Filed: Apr. 19, 2019
(86) PCT No.: PCT/US2019/028327

§ 371 Date: Oct. 19, 2020

Related U.S. Application Data
(60) Provisional application No. 62/660,011, filed on Apr. 19, 2018.

(51) Int. Cl. A61M 25/01; A61M 25/00; A61N 1/36

(52) U.S. Cl. CPC A61M 25/0113; A61N 1/36; A61M 25/0136; A61M 25/0075

(57) ABSTRACT

A medical device is provided that can be held and operated with a single hand to cause a catheter to be placed at a selected location in a patient's body. The user's other hand is free to perform other tasks, such as operating an ultrasound probe, for example. The medical device may also include a valve that can be operated with the same hand that holds the medical device to open a port to cause a fluid, such as a nerve block agent, for example, to be injected into the patient's body at the selected location.

### CROSS-REFERENCE TO RELATED APPLICATIONS

The present application is a U.S. National Phase filing under 35 U.S.C. § 371 of PCT International Application No. PCT/US2019/028327 filed on Apr. 19, 2019, that claims the benefit of and priority to the filing date of U.S. provisional application entitled “A MEDICAL DEVICE FOR DEPLOYING A CATHETER THAT CAN BE OPERATED WITH A SINGLE HAND, AND A METHOD” having Ser. No. 62/660,011, filed Apr. 19, 2018, the contents of which are incorporated by reference herein in their entireties.

### TECHNICAL FIELD

The present disclosure generally relates to medical devices, and more particularly, to a medical device for deploying a catheter that can be operated with a single hand of the user to allow the user's other hand to be used to perform other tasks, e.g., operating an ultrasound probe.

### BACKGROUND

One of the difficulties associated with current peripheral nerve catheter placement for regional anesthesia (typically for patients having orthopedic procedures, although used in other clinical situations as well) is that it often requires two providers to perform the procedure due to the need to operate an ultrasound device and view the ultrasound image while placing the catheter in order to ensure that the catheter is accurately positioned. For this reason, it is virtually impossible for a single provider to thread the catheter while operating an ultrasound probe and view the procedure in real time under such ultrasound guidance. If the wire is not threaded in the appropriate place, sometimes an entirely new kit and/or wire must be opened in order for a new nerve block catheter to be placed.

Accordingly, a need exists for a medical device for deploying a catheter that can be operated with a single hand.

Furthermore, there remains a need for a medical device that can allow for aspiration and injection with the catheter in position to be deployed; all while freeing up the user's other hand to perform other tasks, such as operating an ultrasound probe, for example.

### SUMMARY

In various aspects, medical devices are provided that overcome one or more of the aforementioned problems. For example, medical devices for deploying a catheter are provided that can be operated with a single hand. Methods of using the devices are also provided.

In some aspects, a medical device is provided that is controllable with a hand of a user in which the medical device is held to deploy a catheter in a patient's body, the medical device having: a housing, the housing having a catheter holder that is configured to hold a catheter, the housing having a first opening formed in a forward end thereof through which a distal end of the catheter passes when the catheter is being deployed, the housing having a catheter pathway along which the catheter moves that extends between the first opening and the catheter holder, the forward end being adapted to be removably coupled with a proximal end of a needle; and a catheter deployment and retrieval mechanism mechanically coupled with the housing and configured to operate in a catheter deployment mode and in a catheter retrieval mode, wherein in the catheter deployment mode, the catheter deployment and retrieval mechanism is controllable with a digit of a hand of the user to cause the catheter to be fed out of the catheter holder and to move along the catheter pathway in a first direction, wherein in the catheter retrieval mode, the catheter deployment and retrieval mechanism is controllable with a digit of a hand of the user to cause the catheter to be retracted and to move along the catheter pathway in a second direction that is substantially opposite the first direction.

In some aspects, a catheter gun is provided for deploying and retracting a catheter, wherein the catheter gun is controllable with a first hand of a user in which the catheter gun is held while operating an ultrasound probe with a second hand of the user, the catheter gun having: a housing, the housing having a catheter holder that is configured to hold a catheter in a coiled configuration, the housing having a first opening formed in a forward end thereof through which a distal end of the catheter passes when the catheter is being deployed, the housing having a catheter pathway along which the catheter moves that extends between the first opening and the catheter holder, the forward end being adapted to be removably coupled with a proximal end of a needle, the housing having a handle adapted to be gripped by a user; and a catheter deployment and retrieval mechanism being configured to operate in a catheter deployment mode and in a catheter retrieval mode, wherein when user grips the handle with the first hand, a digit of the first hand can reach a trigger-like device of the catheter deployment and retrieval mechanism, wherein in the catheter deployment mode, the trigger-like device is controllable with a digit of the first hand to cause the catheter to be fed out of the catheter holder and to move along the catheter pathway in a first direction as the user operates the ultrasound probe with the second hand, wherein in the catheter retrieval mode, the trigger-like device is controllable with a digit of the first hand to cause the catheter to be retracted and to move along the catheter pathway in a second direction as the user operates the ultrasound probe with the second hand, the first direction being substantially opposite the second direction.

In some aspects, a method is provided for controlling a medical device with a hand of a user to deploy a catheter in a patient's body, the method including: with a hand of the user, moving the medical device to cause a distal end of a needle to be inserted into a first location of the patient's body, wherein a proximal end of the needle is mechanically coupled by a locking mechanism to a forward end of the medical device; with a digit of a hand of the user, placing a catheter deployment and retrieval mechanism of the medical device in a catheter deployment mode of operations, the catheter being held in a coiled configuration in a catheter holder of the medical device; and with a digit of a hand of the user, operating a trigger-like device of the catheter deployment and retrieval mechanism to cause the catheter to be fed out of the catheter holder and to move along the catheter pathway in a first direction.

Other systems, methods, features, and advantages of medical devices and methods will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Further aspects of the present disclosure will be readily appreciated upon review of the detailed description of its various aspects, described below, when taken in conjunction with the accompanying drawings.

FIG. 1 is a side view of a first exemplary medical device in accordance with various aspects of the disclosure.

### DETAILED DESCRIPTION

In various aspects, medical device is provided that can be held and operated with a single hand to cause a catheter to be placed at a selected location in a patient's body. The user's other hand is free to perform other tasks, such as operating an ultrasound probe, for example.

Before the present disclosure is described in greater detail, it is to be understood that this disclosure is not limited to particular aspects described, and as such may, of course, vary. It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant specification should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

Although any methods and materials similar or equivalent to those described herein can also be used in the practice or testing of the present disclosure, the preferred methods and materials are now described. Functions or constructions well-known in the art may not be described in detail for brevity and/or clarity. Aspects and features of the present disclosure will employ, unless otherwise indicated, medical and surgical techniques, as well as techniques for the design and manufacture of medical devices and surgical tools and the like, which are within the skill of the art. Such techniques are explained fully in the literature.

It should be noted that ratios, concentrations, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In some aspects, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

In some instances, units may be used herein that are non-metric or non-SI units. Such units may be, for instance, in U.S. Customary Measures, e.g., as set forth by the National Institute of Standards and Technology, Department of Commerce, United States of America in publications such as NIST HB 44, NIST HB 133, NIST SP 811, NIST SP 1038, NBS Miscellaneous Publication 214, and the like. The units in U.S. Customary Measures are understood to include equivalent dimensions in metric and other units (e.g., a dimension disclosed as “1 inch” is intended to mean an equivalent dimension of “2.5 cm”; a unit disclosed as “1 pcf” is intended to mean an equivalent dimension of 0.157 kN/m³; or a unit disclosed 100° F. is intended to mean an equivalent dimension of 37.8° C.; and the like) as understood by a person of ordinary skill in the art.

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

The articles “a” and “an,” as used herein, mean one or more when applied to any feature in aspects of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used.

In accordance with various aspects of the disclosure, the medical device can include a housing and a catheter deployment and retrieval mechanism. The housing can have a catheter holder that is configured to hold a catheter, preferably, but not necessarily, in a coiled configuration. A first opening is formed in a forward end of the housing through which a distal end of the catheter passes when the catheter is being deployed. The catheter deployment and retrieval mechanism is mechanically coupled to the housing and to the catheter and is controllable with a digit of a hand of the user to cause the catheter to be fed out of the catheter holder and to move along a catheter pathway of the housing toward a deployment location in the patient's body. The forward end of the housing is adapted to be removably coupled with a proximal end of a needle, such as a tuohy needle, for example.

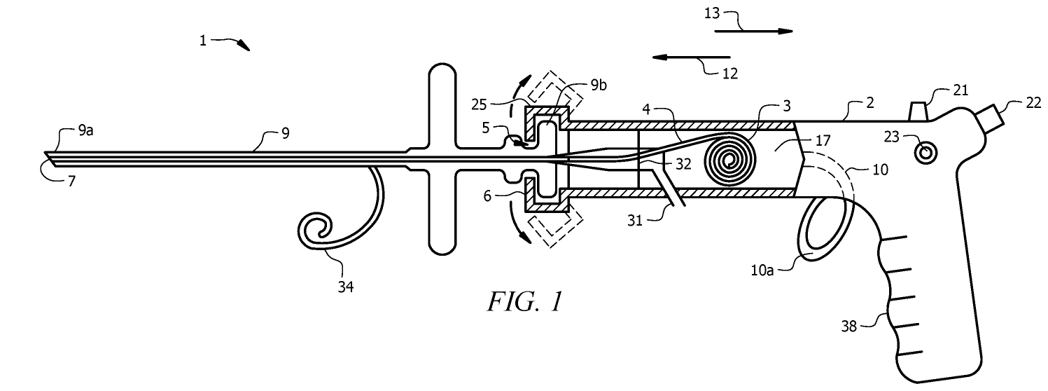

A first exemplary medical device will now be described with reference to FIG. 1. It should be noted that many variations can be made to the aspects of FIG. 1 within the scope of the present disclosure. The first exemplary device shown in FIG. 1 is one example of the medical device in accordance with the inventive principles and concepts that can be easily held in one hand and operated with the same hand to cause a catheter to be deployed or retracted/retrieved if necessary during the performance of the procedure. As will be understood by those skilled in the art in view of the description provided herein, many modifications may be made to the first exemplary medical device shown in FIG. 1 while still achieving the goals of the invention.

FIG. 1 is a side view of a first exemplary medical device 1 in accordance with various aspects of the disclosure. The medical device 1 is controllable with a hand of a user in which the medical device 1 is held to deploy a catheter in a patient's body. This frees the user's other hand to perform other tasks, such as operating an ultrasound probe, for example. The housing 2 of the medical device 1 has a catheter holder 3 that is configured to hold a catheter 4, preferably, but not necessarily, in a coiled configuration. A first opening 5 formed in a forward end 6 of the housing 2 is adapted to allow a distal end 7 of the catheter 4 to pass when the catheter 4 is being deployed, or placed, at a selected location in the patient's body. Within the housing 2, the catheter 4 moves along a catheter pathway that extends between the first opening 5 and the catheter holder 3. The forward end 6 of the housing 2 is adapted to be removably coupled with a proximal end of a needle 9, which may be, for example, a peripheral nerve block needle.

A catheter deployment and retrieval mechanism 10 of the medical device 1 is mechanically coupled with the housing 2 and is configured to operate in a catheter deployment mode and in a catheter retrieval mode. In the catheter deployment mode, the catheter deployment and retrieval mechanism 10 is controllable with a digit of a hand of the user to cause the catheter 4 to be fed out of the catheter holder 3 and to move along the catheter pathway in a first direction 12. As indicated above, this frees the user's other hand to perform other tasks, such as operating an ultrasound probe. In the catheter retrieval mode, the catheter deployment and retrieval mechanism 10 is controllable with a digit of the user's hand to cause the catheter to be retracted and to move along the catheter pathway in a second direction 13. This allows the catheter to be repositioned to a different location in the patient's body. For example, the user may retract the catheter 4, insert the distal end 9a of the needle 9 at a different location in the patient's body, and redeploy the catheter 4 at the new location. Moreover, this can be done as the user operates an ultrasound probe with the other hand. As can be seen in FIG. 1, the first direction 12 is substantially opposite the second direction 13.

The medical device 1 has a handle 38 that is adapted to be gripped by a hand of the user. In accordance with these aspects, the catheter deployment and retrieval mechanism 10 includes a trigger-like device 10a that the user pulls with a digit of the hand to operate the mechanism 10 in the catheter deployment mode and pushes with a digit of the hand to operate the mechanism 10 in the catheter retrieval mode. The catheter deployment and retrieval mechanism 10 includes elements that grip the catheter 4 and either feed it out in the first direction 12 or retract it in the second direction 13. As will be understood by those of skill in the art, a variety of mechanisms can be constructed to perform these tasks. The inventive principles and concepts are not limited with respect to the configuration of the catheter deployment and retrieval mechanism 10, since a variety of configurations may be used for this purpose. Persons of skill in the art will understand the manner in which various configurations for these purposes can be achieved.

In accordance with another aspect, instead of pulling or pushing the trigger-like device 10a to advance or retract the catheter 4, respectively, the catheter deployment and retrieval mechanism 10 includes a ratchet mechanism that can be switched between the catheter deployment mode and the catheter retrieval mode. When the ratchet mechanism is in the catheter deployment mode, pulling of the trigger-like device 10a with a digit of the hand causes the catheter 4 to move in the first direction 12, and when the ratchet mechanism is in the catheter retrieval mode, pulling of the trigger-like device 10a causes the catheter 4 to move in the second direction 13.

In accordance with various aspects, the catheter holder 3 is a cartridge that is removably secured to a cartridge-mounting surface 17 of the housing and engaged with the catheter deployment and retrieval mechanism 10. A first release mechanism 21 is adapted to be operated by a digit of the hand of the user to release a first locking mechanism that locks the cartridge to cartridge-mounting surface 17 and that places a friction fit on the catheter 4 for deployment. Releasing the first locking mechanism removes the friction grip from the catheter 4 and decouples, or disengages, the cartridge from the medical device 1. The first locking mechanism may include a spring-loaded release that ejects the cartridge away from the cartridge-mounting surface 17. As will be understood by those of skill in the art, a variety of locking mechanisms may be configured to perform these tasks. The inventive principles and concepts are not limited to any particular locking mechanism or release mechanism for performing these functions.

In accordance with various aspects, the medical device 1 includes a second release mechanism 22 that is adapted to be operated by a digit of the hand of the user to cause the proximal end 9b of the needle 9 to decouple from the forward end 6 of the housing 2. In cases where the needle 9 is a tuohy needle, for example, the proximal end 9b of the needle 9 comprises a hub that is locked to the forward end 6 of the housing 2 by a second locking mechanism 25. As will be understood by those of skill in the art, a variety of locking mechanisms may be configured to perform this function. The inventive principles and concepts are not limited to any particular locking mechanism or release mechanism for performing these functions. In accordance with an aspect, the second locking mechanism 25 is a standard lever lock that snaps closed to lock the needle 9 to the medical device 1 and that swings open as indicated by the dashed lines in FIG. 1 when the second release mechanism 22 is activated. Reference numeral 23 represents an alternative location for the either of the release mechanisms 21 and 22.

In accordance with a representative aspect, the medical device 1 has an optional first port 31 mechanically coupled to, or integrally formed in, the housing 2. The first port is adapted to introduce a fluid (e.g., a nerve block fluid) into the housing 2 toward the direction of the needle 9. In accordance with various aspects, the first port 31 is coupled to an optional one-way valve 32 that allows the fluid to flow through it in the first direction 12, while preventing the fluid from flowing back through the valve 32 in the second direction 13. The valve 32 also allows two-way movement of the catheter 4 through the valve 32, i.e., movement in the first and second directions 12 and 13, respectively. In accordance with some aspects, the valve 32 is a diaphragm that acts as a one-way valve to allow fluid introduced into the first port 31 to flow in the first direction 12 toward the proximal end 9b of the needle 9 while preventing the fluid from flowing in the second direction 13. The catheter 4 can move in the first and second directions 12 and 13, respectively, through the diaphragm. As will be understood by those of skill in the art, a variety of valve configurations are suitable for this purpose, and therefore the invention is not limited to any particular valve configuration for performing this function. In some aspects, the medical device 1 does not include the first port 31 or the valve 32.

In accordance with some aspects, the medical device 1 includes an optional nerve stimulating electrically-conductive wire 34 that extends outside of the needle 9 and that is electrically coupled to the catheter. Electrical current can be applied to the distal end 7 of the catheter 4 via the wire 34 for stimulating targeted nerves of the patient. In some aspects, the medical device 1 does not include the nerve stimulating electrically-conductive wire 34.

In accordance with the aspects shown in FIG. 1, the first exemplary medical device 1 has the form of a gun. The medical device 1 includes a handle 38 that is adapted to be gripped by the user. When the user is gripping the medical device 1 by the handle 38, the user can easily pull or push the trigger-like device 10a using the index finger and can easily activate the release mechanisms 21, 22 or 23 using the thumb of the same hand.

The medical device 1 can be used to place the catheter 4 in the following manner, assuming that the user is right-handed and that the user is operating an ultrasound probe with the user's left hand. Gripping the handle 38 of the medical device 1 in the user's right hand, the user inserts the distal end 9a of the needle 9 at a target location on the patient's body while operating the ultrasound probe with the user's left hand to view the position of the distal end 9a of the needle 9 as it is being inserted. Once the needle 9 is in position, the user may optionally cause fluid to be injected through the first port 31 and through the needle 9 into the target location in the patient's body. The user then pulls the trigger-like device 10a to advance the catheter 4 in the first direction 12 into the target location in the patient's body.

Once the distal end 7 of the catheter 4 is at the target location, the user presses the first release mechanism 21 to cause the catheter 4 to be decoupled from the medical device 1 (e.g., to eject the catheter cartridge). The user then presses the second release mechanism 22 to cause the needle 9 to be decoupled from the medical device 1. Once the catheter 4 and the needle 9 have been decoupled from the medical device 1, the medical device 1 may be freely moved without imparting movement to the catheter 4. The needle 9 and catheter 4 can then be manipulated in the same fashion in which indwelling catheters are typically placed in patients. The needle 9 is then removed over the catheter 4 from the patient leaving the catheter 4 inserted in the patient at the desired location.

If a determination is made that the distal end 9a of the needle 9 needs to be moved after the catheter 4 has been advanced into the patient's body, the user can retract the catheter by pushing, rather than pulling the trigger-like device 10a. Once the catheter 4 has been retracted a sufficient distance, the distal end 9a of the needle 9 is inserted into the patient's body at the new target location. The catheter 4 is then deployed by again pulling the trigger-like device 10a. The first release mechanism 21 is then pressed to decouple the catheter 4 from the medical device 1. The second release mechanism 22 is then pressed to decouple the needle 9 from the medical device 1.

It should be noted that the disclosure has been described with respect to illustrative aspects for the purpose of describing the principles and concepts of the disclosure. The invention is not limited to these embodiments. For example, while the device has been described with reference to a particular configuration of the housing 2, the housing 2 can in some aspects have any suitable configuration. Likewise, the catheter deployment and retrieval mechanism 10 may have any suitable configuration. Also, although aspects of the disclosure have been described with reference to the catheter being deployed by passing through the needle, the medical device may instead be configured such that the catheter is deployed by passing over the needle, as will be understood by those of skill in the art in view of the description provided herein. As will be understood by those skilled in the art in view of the description being provided herein, many modifications may be made to the aspects described herein while still achieving the goals of the disclosure, and all such modifications are within the scope of the disclosure and may be described in the claims.

It should be emphasized that the above-described aspects of the present disclosure are merely possible examples of implementations, and are set forth only for a clear understanding of the principles of the disclosure. Many variations and modifications may be made to the above-described aspects of the disclosure without departing substantially from the spirit and principles of the disclosure. All such modifications and variations are intended to be included herein within the scope of this disclosure.

The disclosure will be better understood upon reading the following aspects which should not be confused with the claims. Any of the numbered aspects described below can be, in some but not necessarily all instances, combined with other aspects specifically numbered below and/or other aspects described elsewhere in this disclosure.

Aspect 1. A medical device that is controllable with a hand of a user in which the medical device is held to deploy a catheter in a patient's body, the medical device comprising: a housing, the housing having a catheter holder that is configured to hold a catheter, the housing having a first opening formed in a forward end thereof through which a distal end of the catheter passes when the catheter is being deployed, the housing having a catheter pathway along which the catheter moves that extends between the first opening and the catheter holder, the forward end being adapted to be removably coupled with a proximal end of a needle; and a catheter deployment and retrieval mechanism mechanically coupled with the housing and configured to operate in a catheter deployment mode and in a catheter retrieval mode, wherein in the catheter deployment mode, the catheter deployment and retrieval mechanism is controllable with a digit of a hand of the user to cause the catheter to be fed out of the catheter holder and to move along the catheter pathway in a first direction, wherein in the catheter retrieval mode, the catheter deployment and retrieval mechanism is controllable with a digit of a hand of the user to cause the catheter to be retracted and to move along the catheter pathway in a second direction that is substantially opposite the first direction.

Aspect 2. The medical device of any one of Aspects 1-17, wherein the medical device has a handle adapted to be gripped by a hand of the user, and wherein the catheter deployment and retrieval mechanism includes a trigger-like device that the user pulls with a digit of a hand of the user to operate the catheter deployment and retrieval mechanism in the catheter deployment mode and in the catheter retrieval mode.

Aspect 3. The medical device of any one of Aspects 1-17, wherein the catheter deployment and retrieval mechanism includes a ratchet mechanism that can be switched between the catheter deployment mode and the catheter retrieval mode, wherein when the ratchet mechanism is in the catheter deployment mode, pulling of the trigger-like device causes the catheter to move in the first direction along the catheter pathway, and wherein when the ratchet mechanism is in the catheter retrieval mode, pulling of the trigger-like device causes the catheter to move in the second direction along the catheter pathway.

Aspect 4. The medical device of any one of Aspects 1-17, wherein the medical device has a handle adapted to be gripped by a hand of the user, and wherein the catheter deployment and retrieval mechanism includes a trigger-like device that the user pulls with a digit of a hand of the user to advance the catheter in the first direction in the catheter deployment mode and that the user pushes to retract the catheter in the second direction in the catheter retraction mode.

Aspect 5. The medical device of any one of Aspects 1-17, wherein the catheter holder is a cartridge that holds the catheter in a coiled configuration, the cartridge being removably secured to a cartridge-mounting surface of the housing and being engaged with the catheter deployment and retrieval mechanism.

Aspect 6. The medical device of any one of Aspects 1-17, further comprising: at least a first release mechanism that is adapted to be operated by a digit of a hand of the user to unlock a first locking mechanism that locks the cartridge to the cartridge-mounting surface and places a friction grip on the catheter, wherein operating the first release mechanism decouples the cartridge from the medical device and removes the friction grip.

Aspect 7. The medical device of any one of Aspects 1-17, further comprising: a second release mechanism that is adapted to be operated by a digit of a hand of the user to unlock a second locking mechanism that locks the proximal end of the needle to the forward end of the medical device, wherein operating the second release mechanism decouples the proximal end of the needle from the forward end of the housing.

Aspect 8. The medical device of any one of Aspects 1-17, further comprising: a first port mechanically coupled to, or integrally formed in, the housing, the first port being adapted to introduce a fluid into the housing; and a diaphragm that acts as a one-way valve to allow fluid introduced into the first port to flow in the first flow direction toward the proximal end of the needle while preventing the fluid from flowing in the second flow direction, and wherein the diaphragm allows the catheter to move through the diaphragm in the first and second directions.

Aspect 9. The medical device of any one of Aspects 1-17, further comprising: a nerve stimulating wire of electrically-conductive material extending outside of the needle or elsewhere on the device and being electrically coupled to the catheter or within the catheter.

Aspect 10. A catheter gun for deploying and retracting a catheter, wherein the catheter gun is controllable with a first hand of a user in which the catheter gun is held while operating an ultrasound probe with a second hand of the user, the catheter gun comprising: a housing, the housing having a catheter holder that is configured to hold a catheter in a coiled configuration, the housing having a first opening formed in a forward end thereof through which a distal end of the catheter passes when the catheter is being deployed, the housing having a catheter pathway along which the catheter moves that extends between the first opening and the catheter holder, the forward end being adapted to be removably coupled with a proximal end of a needle, the housing having a handle adapted to be gripped by a user; and a catheter deployment and retrieval mechanism being configured to operate in a catheter deployment mode and in a catheter retrieval mode, wherein when user grips the handle with the first hand, a digit of the first hand can reach a trigger-like device of the catheter deployment and retrieval mechanism, wherein in the catheter deployment mode, the trigger-like device is controllable with a digit of the first hand to cause the catheter to be fed out of the catheter holder and to move along the catheter pathway in a first direction as the user operates the ultrasound probe with the second hand, wherein in the catheter retrieval mode, the trigger-like device is controllable with a digit of the first hand to cause the catheter to be retracted and to move along the catheter pathway in a second direction as the user operates the ultrasound probe with the second hand, the first direction being substantially opposite the second direction.

Aspect 11. The catheter gun of any one of Aspects 1-17, wherein the catheter deployment and retrieval mechanism includes a ratchet mechanism that can be placed switched between the catheter deployment mode and the catheter retrieval mode, wherein when the ratchet mechanism is in the catheter deployment mode, pulling of the trigger-like device with a digit of the first hand causes the catheter to move in the first direction along the catheter pathway, and wherein when the ratchet mechanism is in the catheter retrieval mode, pulling of the trigger-like device with a digit of the first hand causes the catheter to move in the second direction along the catheter pathway.

Aspect 12. The catheter gun of any one of Aspects 1-17, wherein pulling the trigger-like device with a digit of the first hand of the user advances the catheter in the first direction in the catheter deployment mode and pushing the trigger-like device retracts the catheter in the second direction in the catheter retraction mode.

Aspect 13. The catheter gun of any one of Aspects 1-17, wherein the catheter holder is a cartridge that is removably secured to a cartridge-mounting surface of the housing and engaged with the catheter deployment and retrieval mechanism.

Aspect 14. The catheter gun of any one of Aspects 1-17, further comprising: at least a first release mechanism that is adapted to be operated by a digit of the first hand of the user to unlock a first locking mechanism that locks the cartridge to the cartridge-mounting surface and places a friction grip on the catheter, wherein operating the first release mechanism decouples the cartridge from the medical device and removes the friction grip.

Aspect 15. The catheter gun of any one of Aspects 1-17, further comprising: a second release mechanism that is adapted to be operated by a digit of the first hand of the user to unlock a second locking mechanism that locks the proximal end of the needle to the forward end of the medical device, wherein operating the second release mechanism decouples the proximal end of the needle from the forward end of the housing.

Aspect 16. The catheter gun of any one of Aspects 1-17, further comprising: a first port mechanically coupled to, or integrally formed in, the housing, the first port being adapted to introduce a fluid through the housing; and a diaphragm that acts as a one-way valve to allow fluid introduced into the first port to flow in a first flow direction toward the proximal end of the needle while preventing the fluid from flowing in a second flow direction that is opposite the first flow direction.

Aspect 17. The catheter gun of any one of Aspects 1-17, further comprising: a nerve stimulating wire of electrically-conductive material extending outside of the needle or the device and being electrically coupled to or within the catheter.

Aspect 18. A method for controlling a medical device with a hand of a user to deploy a catheter in a patient's body, the method comprising: with a hand of the user, moving the medical device to cause a distal end of a needle to be inserted into a first location of the patient's body, wherein a proximal end of the needle is mechanically coupled by a locking mechanism to a forward end of the medical device; with a digit of a hand of the user, placing a catheter deployment and retrieval mechanism of the medical device in a catheter deployment mode of operations, the catheter being held in a coiled configuration in a catheter holder of the medical device; and

with a digit of a hand of the user, operating a trigger-like device of the catheter deployment and retrieval mechanism to cause the catheter to be fed out of the catheter holder and to move along the catheter pathway in a first direction.

Aspect 19. The method any one of Aspects 18-22, further comprising: with a digit of a hand of the user, operating the trigger-like device to cause the catheter to be retracted toward the catheter holder and to move along the catheter pathway in a second direction that is substantially opposite to the first direction; with a hand of the user, moving the medical device to cause the distal end of the needle to be removed from the first location of the patient's body and to cause the distal end of the needle to be inserted into a second location of the patient's body; and with a digit of a hand of the user, operating the trigger-like device of the catheter deployment and retrieval mechanism to cause the catheter to be fed out of the catheter holder and to move along the catheter pathway in the first direction.

Aspect 20. The method of any one of Aspects 18-22, further comprising: with a digit of a hand of the user, operating a first release mechanism that causes the catheter holder to be decoupled from a housing of the medical device.

Aspect 21. The method of any one of Aspects 18-22, further comprising: with a digit of a hand of the user, operating a second release mechanism that causes the proximal end of the needle to be decoupled from the forward end of the medical device.

Aspect 22. The method of any one of Aspects 18-21, wherein the method is performed with a device according to any one of Aspects 1-17.

1. A medical device that is controllable with a hand of a user in which the medical device is held to deploy a catheter in a patient's body, the medical device comprising:
a housing, the housing having a catheter holder that is configured to hold a catheter, the housing having a first opening formed in a forward end thereof through which a distal end of the catheter passes when the catheter is being deployed, the housing having a catheter pathway along which the catheter moves that extends between the first opening and the catheter holder, the forward end being adapted to be removably coupled with a proximal end of a needle; and
a catheter deployment and retrieval mechanism mechanically coupled with the housing and configured to operate in a catheter deployment mode and in a catheter retrieval mode, wherein in the catheter deployment mode, the catheter deployment and retrieval mechanism is controllable with a digit of a hand of the user to cause the catheter to be fed out of the catheter holder and to move along the catheter pathway in a first direction, wherein in the catheter retrieval mode, the catheter deployment and retrieval mechanism is controllable with a digit of a hand of the user to cause the catheter to be retracted and to move along the catheter pathway in a second direction that is substantially opposite the first direction.

2. The medical device of claim 1, wherein the medical device has a handle adapted to be gripped by a hand of the user, and wherein the catheter deployment and retrieval mechanism includes a trigger-like device that the user pulls with a digit of a hand of the user to operate the catheter deployment and retrieval mechanism in the catheter deployment mode and in the catheter retrieval mode.

3. The medical device of claim 2, wherein the catheter deployment and retrieval mechanism includes a ratchet mechanism that can be switched between the catheter deployment mode and the catheter retrieval mode, wherein when the ratchet mechanism is in the catheter deployment mode, pulling of the trigger-like device causes the catheter to move in the first direction along the catheter pathway, and wherein when the ratchet mechanism is in the catheter retrieval mode, pulling of the trigger-like device causes the catheter to move in the second direction along the catheter pathway.

4. The medical device of claim 1, wherein the medical device has a handle adapted to be gripped by a hand of the user, and wherein the catheter deployment and retrieval mechanism includes a trigger-like device that the user pulls with a digit of a hand of the user to advance the catheter in the first direction in the catheter deployment mode and that the user pushes to retract the catheter in the second direction in the catheter retraction mode.

5. The medical device of claim 1, wherein the catheter holder comprises a cartridge that holds the catheter in a coiled configuration, the cartridge being removably secured to a cartridge-mounting surface of the housing and being engaged with the catheter deployment and retrieval mechanism.

6. The medical device of claim 5, further comprising:
at least a first release mechanism that is adapted to be operated by a digit of a hand of the user to unlock a first locking mechanism that locks the cartridge to the cartridge-mounting surface and places a friction grip on the catheter, wherein operating the first release mechanism decouples the cartridge from the medical device and removes the friction grip.

7. The medical device of claim 6, further comprising:
a second release mechanism that is adapted to be operated by a digit of a hand of the user to unlock a second locking mechanism that locks the proximal end of the needle to the forward end of the medical device, wherein operating the second release mechanism decouples the proximal end of the needle from the forward end of the housing.

8. The medical device of claim 1, further comprising:
a first port mechanically coupled to, or integrally formed in, the housing, the first port being adapted to introduce a fluid into the housing; and
a diaphragm that acts as a one-way valve to allow fluid introduced into the first port to flow in the first flow direction toward the proximal end of the needle while preventing the fluid from flowing in the second flow direction, and wherein the diaphragm allows the catheter to move through the diaphragm in the first and second directions.

9. The medical device of claim 1, further comprising:
a nerve stimulating wire of electrically-conductive material having a proximal end that is exposed to allow an electrical current to be applied to the proximal end of the wire and having a distal end that is electrically coupled to the catheter.

10. A catheter gun for deploying and retracting a catheter, wherein the catheter gun is controllable with a first hand of a user in which the catheter gun is held while operating an ultrasound probe with a second hand of the user, the catheter gun comprising:
a housing, the housing having a catheter holder that is configured to hold a catheter in a coiled configuration, the housing having a first opening formed in a forward end thereof through which a distal end of the catheter passes when the catheter is being deployed, the housing having a catheter pathway along which the catheter moves that extends between the first opening and the catheter holder, the forward end being adapted to be removably coupled with a proximal end of a needle, the housing having a handle adapted to be gripped by a user; and
a catheter deployment and retrieval mechanism being configured to operate in a catheter deployment mode and in a catheter retrieval mode, wherein when user grips the handle with the first hand, a digit of the first hand can reach a trigger-like device of the catheter deployment and retrieval mechanism, wherein in the catheter deployment mode, the trigger-like device is controllable with a digit of the first hand to cause the catheter to be fed out of the catheter holder and to move along the catheter pathway in a first direction as the user operates the ultrasound probe with the second hand, wherein in the catheter retrieval mode, the trigger-like device is controllable with a digit of the first hand to cause the catheter to be retracted and to move along the catheter pathway in a second direction as the user operates the ultrasound probe with the second hand, the first direction being substantially opposite the second direction.

11. The catheter gun of claim 10, wherein the catheter deployment and retrieval mechanism includes a ratchet mechanism that can be placed switched between the catheter deployment mode and the catheter retrieval mode, wherein when the ratchet mechanism is in the catheter deployment mode, pulling of the trigger-like device with a digit of the first hand causes the catheter to move in the first direction along the catheter pathway, and wherein when the ratchet mechanism is in the catheter retrieval mode, pulling of the trigger-like device with a digit of the first hand causes the catheter to move in the second direction along the catheter pathway.

12. The catheter gun of claim 10, wherein pulling the trigger-like device with a digit of the first hand of the user advances the catheter in the first direction in the catheter deployment mode and pushing the trigger-like device retracts the catheter in the second direction in the catheter retraction mode.

13. The catheter gun of claim 10, wherein the catheter holder is a cartridge that is removably secured to a cartridge-mounting surface of the housing and engaged with the catheter deployment and retrieval mechanism.

14. The catheter gun of claim 13, further comprising:
at least a first release mechanism that is adapted to be operated by a digit of the first hand of the user to unlock a first locking mechanism that locks the cartridge to the cartridge-mounting surface and places a friction grip on the catheter, wherein operating the first release mechanism decouples the cartridge from the medical device and removes the friction grip.

15. The catheter gun of claim 14, further comprising:
a second release mechanism that is adapted to be operated by a digit of the first hand of the user to unlock a second locking mechanism that locks the proximal end of the needle to the forward end of the medical device, wherein operating the second release mechanism decouples the proximal end of the needle from the forward end of the housing.

16. The catheter gun of claim 10, further comprising:
a first port mechanically coupled to, or integrally formed in, the housing, the first port being adapted to introduce a fluid through the housing; and
a diaphragm that acts as a one-way valve to allow fluid introduced into the first port to flow in a first flow direction toward the proximal end of the needle while preventing the fluid from flowing in a second flow direction that is opposite the first flow direction.

17. The catheter gun of claim 10, further comprising:
a nerve stimulating wire of electrically-conductive material having a proximal end that is exposed to allow an electrical current to be applied to the proximal end of the wire and having a distal end that is electrically coupled to the catheter.

18. A method for controlling a medical device with a hand of a user to deploy a catheter in a patient's body, the method comprising:
with a hand of the user, moving the medical device to cause a distal end of a needle to be inserted into a first location of the patient's body, wherein a proximal end of the needle is mechanically coupled by a locking mechanism to a forward end of the medical device;
with a digit of a hand of the user, placing a catheter deployment and retrieval mechanism of the medical device in a catheter deployment mode of operations, the catheter being held in a coiled configuration in a catheter holder of the medical device; and
with a digit of a hand of the user, operating a trigger-like device of the catheter deployment and retrieval mechanism to cause the catheter to be fed out of the catheter holder and to move along the catheter pathway in a first direction.

19. The method of claim 18, further comprising:
with a digit of a hand of the user, operating the trigger-like device to cause the catheter to be retracted toward the catheter holder and to move along the catheter pathway in a second direction that is substantially opposite to the first direction;
with a hand of the user, moving the medical device to cause the distal end of the needle to be removed from the first location of the patient's body and to cause the distal end of the needle to be inserted into a second location of the patient's body;
with a digit of a hand of the user, operating the trigger-like device of the catheter deployment and retrieval mechanism to cause the catheter to be fed out of the catheter holder and to move along the catheter pathway in the first direction.

20. The method of claim 18, further comprising:
with a digit of a hand of the user, operating a first release mechanism that causes the catheter holder to be decoupled from a housing of the medical device; and
with a digit of a hand of the user, operating a second release mechanism that causes the proximal end of the needle to be decoupled from the forward end of the medical device.

21. (canceled)
